(19) United States
(12) Patent Application Publication
(10) Pub. No.: US 2017/0339944 A1
(43) Pub. Date: Nov. 30, 2017

(54) METHOD OF PRESERVING ORGANISMS AND APPARATUS THEREOF

(71) Applicant: HUI-HSING YU, Taipei (TW)

(72) Inventor: HUI-HSING YU, Taipei (TW)

(21) Appl. No.: 15/590,026

(22) Filed: May 9, 2017

(30) Foreign Application Priority Data
May 25, 2016 (TW) 105116352
Mar. 10, 2017 (TW) 106107951

(51) Int. Cl. A01N 1/02; A61M 1/16; A61J 1/05; A61M 1/02; A61J 1/14

(52) U.S. Cl. CPC A01N 1/021; A01N 1/0242; A61M 1/169; A01N 1/02; A01N 1/0278; A61J 1/1412; A61M 1/0259; A61J 1/05

(57) ABSTRACT

A simple, natural method of preserving organisms includes sealing biological blood or organisms in a shell filled with resin, curing the resin, and solidifying the shell for a long time to form amber. The biological blood or organisms can be preserved for a long time. Scholars of later generations may research the integrity of the biological blood or organisms of the present days. Otherwise, the biological blood or organisms may be of no researching meaning and purposes due to pollution or oxidation and dehydration. The organisms of the present days may be reconstructed after 100 years by using technologies of that time. Thus, the organisms being extinct due to pollution can be reconstructed. Diversities of organisms on the earth can be maintained. Precious genes of the organisms of the present days can be preserved for thousands years. It contributes greatly to the study of evolution of organisms.

### BACKGROUND OF THE INVENTION

### 1. Field of the Invention

The invention relates to method of preserving organisms and apparatus thereof and more particularly to a method of preserving organisms and apparatus thereof.

### 2. Description of Related Art

Amber is fossilized tree resin. Because it originates as a soft, sticky tree resin, amber sometimes contains animal and plant material as inclusions. In view of this, it is desirable of providing a method of preserving organisms in amber.

### SUMMARY OF THE INVENTION

It is therefore one object of the invention to provide an apparatus of preserving organisms so that the organisms enclosed by the apparatus can be fossilized to form amber.

It is another object of the invention to provide a method of preserving organisms. The method can be performed without adding additives thereto, thereby causing no pollution to the environment.

For achieving above and other objects, the invention provides a method of preserving organisms comprising the steps of preparing a spherical shell made of a hard material, the shell having an internal space and a truncated conic hole through the shell; infusing organisms into the space through the truncated conic hole in a concealed environment filled with nitrogen and expelling air in the space out of the truncated conic hole; inserting a plug into the truncated conic hole to obstruct the truncated conic hole; applying an adhesive on the truncated conic hole to seal the truncated conic hole and form a spherical container; disposing a mold around the spherical container; pouring viscose resin into the mold through a hole of the mold; solidifying the viscose resin to form an enclosure having a shell member; and removing the mold.

For achieving above and other objects, the invention further provides a method of preserving organisms comprising the steps of preparing a spherical shell made of a flexible material or rubber, the shell having an internal space; piercing a hypodermic needle of one syringe through the shell to infuse organisms into the space and piercing a hypodermic needle of the other syringe through the shell to draw air out of the space by manipulating the other syringe in a concealed environment filled with nitrogen; removing the hypodermic needles, with holes formed by the hypodermic needles, being blocked completely to form a spherical container; disposing a mold around the spherical container; pouring viscose resin into the mold through a hole of the mold; solidifying the viscose resin to form an enclosure having a shell member; and removing the mold.

According to a method of the present invention, the organisms include blood, bone marrow, cerebrospinal fluid, stem cells, cells, semen, embryo, fertilized egg, body fluid (saliva, mucus), fish blood, shrimp blood, liquid secreted by insects, and liquid secreted by plants.

According to a method of the present invention, the viscose resin includes natural resin of pines.

According to a method of the present invention, the viscose resin includes artificial resin made by esterification.

According to a method of the present invention, the viscose resin includes rubber of different hardness and transparency, and silicone of different hardness and transparency.

For achieving above and other objects, the invention still further provides an apparatus of preserving organisms comprising a spherical container, including spherical shell, having an internal space, filled with organisms; and an enclosure sealingly disposed on the spherical container.

According to an embodiment of the present invention, the spherical container is formed by preparing spherical shell made of hard material, the shell having an internal space and truncated conic hole through the shell, infusing organisms into the space through the truncated conic hole and expelling air in the space out of the truncated conic hole, and sealing the truncated conic hole; and wherein the spherical container is formed by preparing spherical shell made of flexible material or rubber, the shell having an internal space, piercing hypodermic needle of one syringe through the shell to infuse organisms into the space and piercing hypodermic needle of the other syringe through the shell to draw air out of the space by manipulating the other syringe, and removing the hypodermic needles, with holes formed by the hypodermic needles, being blocked completely.

According to an embodiment of the present invention, the enclosure is formed by solidifying viscose resin, and the enclosure will be fossilized to form amber after predetermined years.

According to an embodiment of the present invention, the organisms include blood, bone marrow, cerebrospinal fluid, stem cells, cells, semen, embryo, fertilized egg, body fluid (saliva, mucus), fish blood, shrimp blood, liquid secreted by insects, and liquid secreted by plants.

According to an embodiment of the present invention, the viscose resin includes natural resin of pines.

According to an embodiment of the present invention, the viscose resin includes artificial resin made by esterification.

According to an embodiment of the present invention, the viscose resin includes rubber of different hardness and transparency, and silicone of different hardness and transparency.

The above and other objects, features and advantages of the invention will become apparent from the following detailed description taken with the accompanying drawings.

### BRIEF DESCRIPTION OF THE DRAWINGS

FIG. 1 is a sectional view of an apparatus of preserving organisms according to a first preferred embodiment of the invention;

FIG. 2 is a sectional view of the spherical container;

FIG. 3 is a longitudinal sectional view showing blood being transfused into the spherical container;

FIG. 4 is a longitudinal sectional view showing the forming of the enclosure;

FIG. 5 is a sectional view of an apparatus of preserving organisms according to a first preferred embodiment of the invention;

FIG. 6 is a sectional view of the spherical container of FIG. 5;

FIG. 7 is a longitudinal sectional view showing blood being transfused into the spherical container of FIG. 5;

FIG. 8 is a longitudinal sectional view showing the forming of the enclosure of FIG. 5;

FIG. 9 is a longitudinal sectional view showing the apparatus of preserving organisms disposed in the soil;

FIG. 10 is a front view showing the apparatus of preserving organisms made into a pendant; and

FIG. 11 is a perspective view showing the apparatus of preserving organisms made into a ring.

### DETAILED DESCRIPTION OF THE INVENTION

Organisms described by the invention include blood, bone marrow, semen, cerebrospinal fluid, stem cells, cells, saliva, and hair. Specifically, the organism is human blood.

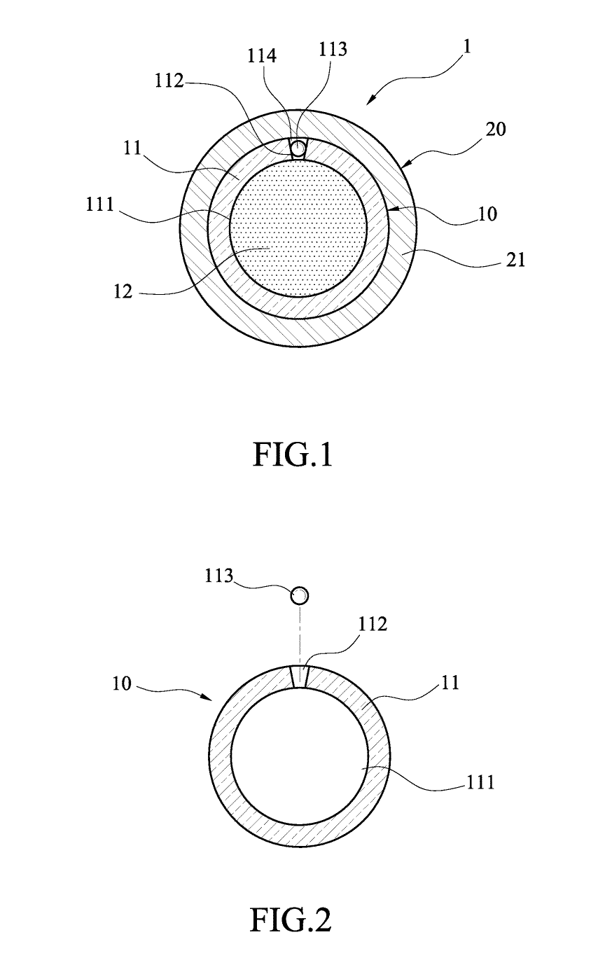

Referring to FIGS. 1 to 4, an apparatus 1 of preserving organisms and method thereof in accordance with a first preferred embodiment of the invention are discussed in detail below.

The apparatus 1 comprises a spherical container 10 and an enclosure 20. The spherical container 10 includes a shell 11 having an internal space 111 and a truncated conic hole 112 through the shell 11. Blood 12 can be infused into the space 111 through the truncated conic hole 112. Thereafter, a spherical plug 113 is inserted into the truncated conic hole 112 to obstruct the truncated conic hole 112. Next, an adhesive 114 is applied on the mouth of the truncated conic hole 112 to seal the truncated conic hole 112. Next, the enclosure 20 includes a shell member 21 formed on the spherical container 10. As a result, the blood 12 can be preserved for a long time. The apparatus 1 can be disposed in the soil 50 (see FIG. 9). The apparatus 1 will be fossilized to form amber after several years.

It is noted that the shell 11 is made of a hard material and has an outer diameter of 0.8 cm. A diameter of the space 111 is 0.4 cm. The raw material of the shell member 21 is viscose resin 21A and can be solidified after filling on an outer surface of the spherical container 10.

A method of preserving organisms according to the first preferred embodiment of the invention is illustrated below and comprises the following steps:

Step (A): Preparing a spherical shell 11 made of a hard material, the shell 11 having an outer diameter of 0.8 cm and an inner diameter of 0.4 cm, i.e., the diameter of a space 111, driving a truncated conic hole 112 through the shell 11.

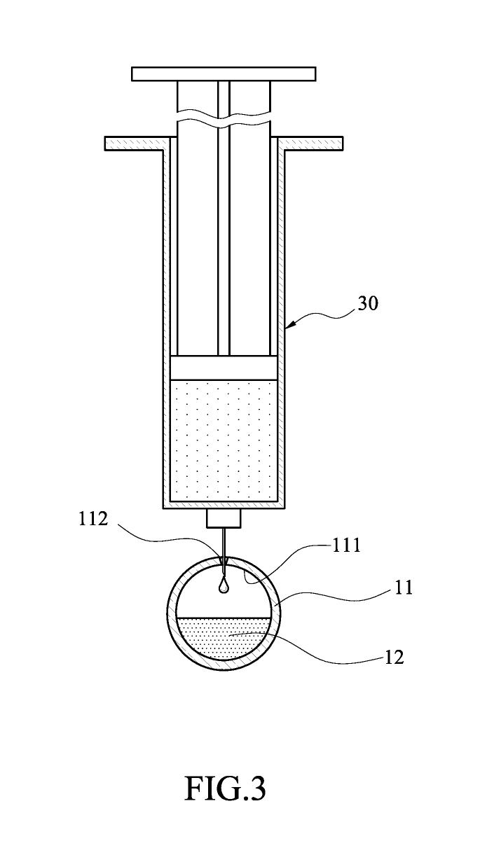

Step (B): As shown in FIG. 3, using a syringe 30 to draw blood 12 thereinto, infusing the blood 12 into the space 111 through the truncated conic hole 112 in a concealed environment filled with nitrogen and expelling air in the space 111 out of the truncated conic hole 112.

Step (C): Inserting a spherical plug 113 into the truncated conic hole 112 to obstruct the truncated conic hole 112.

Step (D): Applying an adhesive 114 on the mouth of the truncated conic hole 112 to seal the truncated conic hole 112 so as to form a spherical container 10.

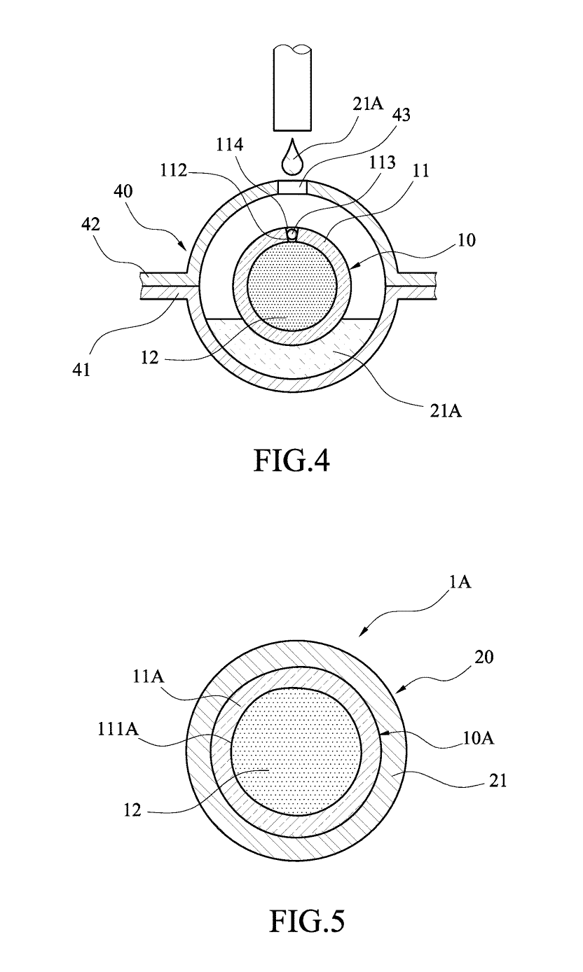

Step (E): As shown in FIG. 4, disposing a mold 40 having an outer diameter of 1.2 cm and including a lower shell 41 and an upper shell 42 around the spherical container 10.

Step (F): Pouring the viscose resin 21A into the mold 40 through a hole 43 until a space between the mold 40 and the spherical container 10 is completely filled with the viscose resin 21A.

Step (G): Waiting a predetermined period of time until the viscose resin 21A solidifies to form an enclosure 20 having a shell member 21; and

Step (H): Removing the mold 40.

It is noted that the step (B) should be performed in a concealed environment filled with nitrogen in order to prevent oxygen from entering the spherical container 10 to contaminate blood 12 stored therein.

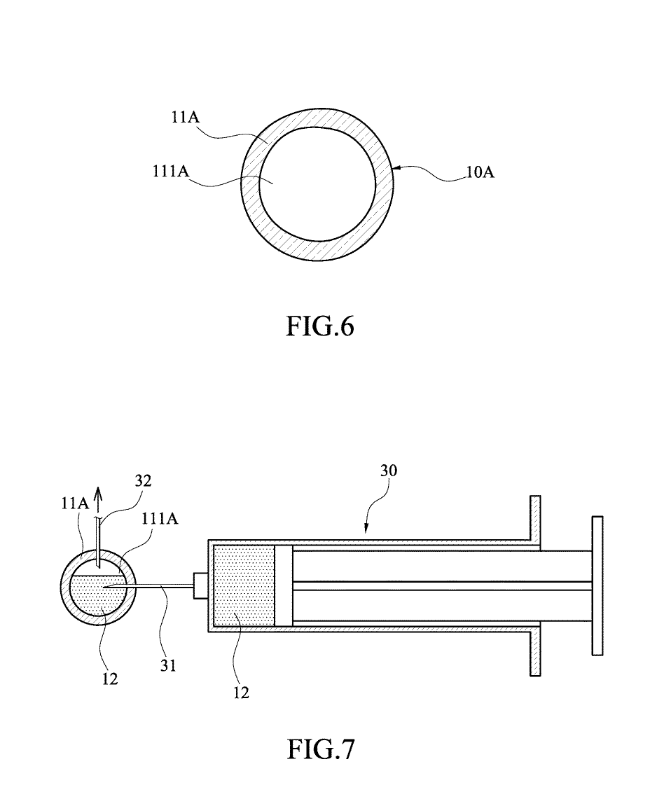

Referring to FIGS. 5 to 8, an apparatus 1A of preserving organisms and method thereof in accordance with a second preferred embodiment of the invention are discussed in detail below. The characteristics of the second preferred embodiment are substantially the same as that of the first preferred embodiment except the following:

The apparatus 1A comprises a spherical container 10A and an enclosure 20. The spherical container 10A includes a shell 11A having an internal space 111A. Blood 12 can be infused into the space 111A by piercing a hypodermic needle 31 of one syringe 30 through the shell 11A and at the same time air in the space 111A can be expelled by piercing a hypodermic needle 32 of the other syringe (not shown) through the shell 11A and drawing air out of the space 111A by manipulating the other syringe. The blood infusion can be stopped when the space 111A is filled with the blood 12. The holes formed by the piercing needles can be blocked completely after removing the needles 31 and 32 due to the nature of the flexible material of the shell 11A. Next, the enclosure 20 includes a shell member 21 formed on the spherical container 10A. As a result, the blood 12 can be preserved for a long time. The apparatus 1A can be disposed in the soil 50 (see FIG. 9). The apparatus 1A will be fossilized to form amber after several years.

It is noted that the shell 11A is made of a flexible material and has an outer diameter of 0.8 cm. A diameter of the space 111A is 0.4 cm. The raw material of the shell member 21 is viscose resin 21A and can be solidified after filling on an outer surface of the spherical container 10A.

A method of preserving organisms according to the second preferred embodiment of the invention is illustrated below and comprises the following steps:

Step (A): Preparing a spherical shell 11A made of a flexible material or rubber, the shell 11A having an outer diameter of 0.8 cm and an inner diameter of 0.4 cm, i.e., the diameter of a space 111A;

Step (B): Using one syringe 30 to draw blood 12 thereinto, infusing the blood 12 into the space 111A by piercing a hypodermic needle 31 of one syringe 30 through the shell 11A and at the same time piercing a hypodermic needle 32 of the other syringe (not shown) through the shell 11A to draw air out of the space 111A by manipulating the other syringe, and stopping the blood infusion when the space 111A is filled with the blood 12;

Step (C): Removing the needles 31 and 32 due to the nature of the flexible material of the shell 11A, in which the holes formed by the piercing needles can be blocked completely to form a spherical container 10A;

Step (D): Disposing a mold 40 having an outer diameter of 1.2 cm and including a lower shell 41 and an upper shell 42 around the spherical container 10A;

Step (E): Pouring the viscose resin 21A into the mold 40 through a hole 43 until a space between the mold 40 and the spherical container 10A is completely filled with the viscose resin 21A;

Step (F): Waiting a predetermined period of time until the viscose resin 21A solidifies to form an enclosure 20 having a shell member 21; and

Step (G): Removing the mold 40.

It is noted that the step (B) should be performed in a concealed environment filled with nitrogen in order to prevent oxygen from entering the spherical container 10A to contaminate blood 12 stored therein.

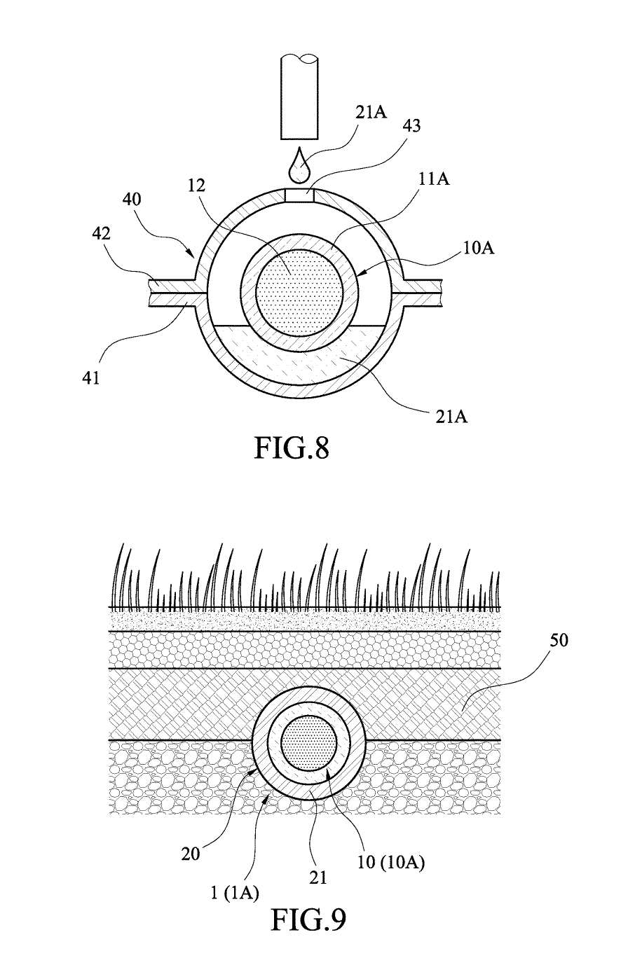

Referring to FIG. 9 in conjunction with FIGS. 4 and 8, the enclosure 20 having a shell member 21 formed by solidifying the viscose resin 21A as a part of the apparatus 1 or 1A will be fossilized to form amber after several years.

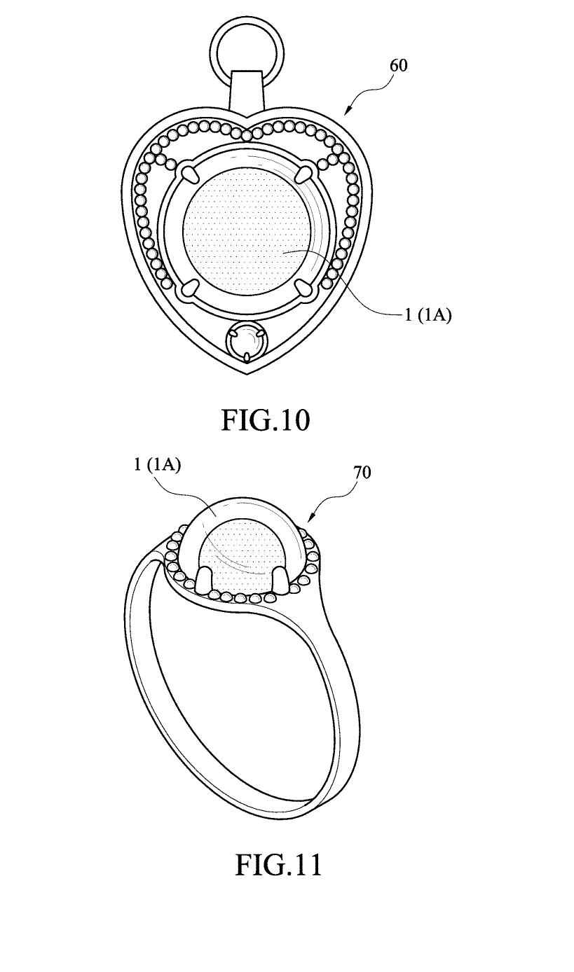

Referring to FIG. 10, the apparatus 1 or 1A is made into a pendant 60.

Referring to FIG. 11, the apparatus 1 or 1A is made into a ring 70.

The human blood 12 can be replaced with animal's bone marrow, bone marrow, cerebrospinal fluid, stem cells, cells, semen, embryo, fertilized egg, body fluid (saliva, mucus), blood of fish, blood of shrimp, insect, or tree resin in other embodiments.

It is noted that one object of the method of preserving organisms and apparatus thereof of the invention is to preserve samples of blood or organisms for a long time so that scholars of later generations may research the integrity of the biological blood and living organisms of the present days. Otherwise, the biological blood or organisms may be of no researching meaning and purposes due to pollution or oxidation and dehydration.

The spherical shell 11 or 11A can be replaced with an oval, a disc-shaped, a ring-shaped, or any of other shapes in other embodiments.

It is noted that resin as the materials of the containers 10, 10A and the shells 11, 11A or the enclosure 20 on each of the containers 10, 10A and the shells 11, 11A is chosen from natural resin of pines. Moreover, the resin can be chosen from natural resin from natural trees, artificial resin made by esterification, rubber of different hardness and transparency, and silicone of different hardness and transparency.

While the invention has been described in terms of preferred embodiments, those skilled in the art will recognize that the invention can be practiced with modifications within the spirit and scope of the appended claims.

What is claimed is:

1. A method of preserving organisms comprising the steps of:
(A) preparing a spherical shell (11) made of a hard material, the shell (11) having an internal space (111) and a truncated conic hole (112) through the shell (11);
(B) infusing organisms (12) into the space (111) through the truncated conic hole (112) in a concealed environment filled with nitrogen and expelling air in the space (111) out of the truncated conic hole (112);
(C) inserting a plug (113) into the truncated conic hole (112) to obstruct the truncated conic hole (112);
(D) applying an adhesive (114) on the truncated conic hole (112) to seal the truncated conic hole (112) and form a spherical container (10);
(E) disposing a mold (40) around the spherical container (10);
(F) pouring viscose resin (21A) into the mold (40) through a hole (43) of the mold (40);
(G) solidifying the viscose resin (21A) to form an enclosure (20) having a shell member (21); and
(H) removing the mold (40).

2. A method of preserving organisms comprising the steps of:
(A) preparing a spherical shell (11A) made of a flexible material or rubber, the shell (11A) having an internal space (111A);
(B) piercing a hypodermic needle (31) of one syringe (30) through the shell (11A) to infuse organisms (12) into the space (111A) and piercing a hypodermic needle (32) of the other syringe (30) through the shell (11A) to draw air out of the space (111A) by manipulating the other syringe (30) in a concealed environment filled with nitrogen;
(C) removing the hypodermic needles (31, 32) with holes formed by the hypodermic needles (31, 32) being blocked completely to form a spherical container (10A);
(D) disposing a mold (40) around the spherical container (10A);
(E) pouring viscose resin (21A) into the mold (40) through a hole (43) of the mold (40);
(F) solidifying the viscose resin (21A) to form an enclosure (20) having a shell member (21); and
(G) removing the mold (40).

3. An apparatus of preserving organisms comprising:
a spherical container (10, 10A) including a spherical shell (11, 11A) having an internal space (111, 111A) filled with organisms (12); and
an enclosure (20) sealingly disposed on the spherical container (10, 10A).

4. The apparatus of preserving organisms of claim 3, wherein the spherical container (10) is formed by preparing a spherical shell (11) made of a hard material, the shell (11) having an internal space (111) and a truncated conic hole (112) through the shell (11), infusing organisms (12) into the space (111) through the truncated conic hole (112) and expelling air in the space (111) out of the truncated conic hole (112), and sealing the truncated conic hole (112); and wherein the spherical container (10A) is formed by preparing a spherical shell (11A) made of a flexible material or rubber, the shell (11A) having an internal space (111A), piercing a hypodermic needle (31) of one syringe (30) through the shell (11A) to infuse organisms (12) into the space (111A) and piercing a hypodermic needle (32) of the other syringe (30) through the shell (11A) to draw air out of the space (111A) by manipulating the other syringe (30), and removing the hypodermic needles (31, 32) with holes formed by the hypodermic needles (31, 32) being blocked completely.

5. The apparatus of preserving organisms of claim 3, wherein the enclosure (20) is formed by solidifying a viscose resin (21A), and the enclosure (20) will be fossilized to form amber after predetermined years.

6. The method of preserving organisms of claim 1, wherein the organisms (12) include blood, bone marrow, cerebrospinal fluid, stem cells, cells, semen, embryo, fertilized egg, body fluid (saliva, mucus), fish blood, shrimp blood, liquid secreted by insects, and liquid secreted by plants.

7. The method of preserving organisms of claim 2, wherein the organisms (12) include blood, bone marrow, cerebrospinal fluid, stem cells, cells, semen, embryo, fertilized egg, body fluid (saliva, mucus), fish blood, shrimp blood, liquid secreted by insects, and liquid secreted by plants.

8. The apparatus of preserving organisms of claim 3, wherein the organisms (12) include blood, bone marrow, cerebrospinal fluid, stem cells, cells, semen, embryo, fertilized egg, body fluid (saliva, mucus), fish blood, shrimp blood, liquid secreted by insects, and liquid secreted by plants.

9. The method of preserving organisms of claim 1, wherein the viscose resin (21A) includes natural resin of pines.

10. The method of preserving organisms of claim 2, wherein the viscose resin (21A) includes natural resin of pines.

11. The apparatus of preserving organisms of claim 3, wherein the viscose resin (21A) includes natural resin of pines.

12. The method of preserving organisms of claim 1, wherein the viscose resin (21A) includes artificial resin made by esterification.

13. The method of preserving organisms of claim 2, wherein the viscose resin (21A) includes artificial resin made by esterification.

14. The apparatus of preserving organisms of claim 3, wherein the viscose resin (21A) includes artificial resin made by esterification.

15. The method of preserving organisms of claim 1, wherein the viscose resin (21A) includes rubber of different hardness and transparency, and silicone of different hardness and transparency.

16. The method of preserving organisms of claim 2, wherein the viscose resin (21A) includes rubber of different hardness and transparency, and silicone of different hardness and transparency.

17. The apparatus of preserving organisms of claim 3, wherein the viscose resin (21A) includes rubber of different hardness and transparency, and silicone of different hardness and transparency.
